(19) United States
(12) Patent Application Publication
(10) Pub. No.: US 2019/0299205 A1
(43) Pub. Date: Oct. 3, 2019

(54) DETECTION DEVICE

(71) Applicant: Chang Gung Memorial Hospital, Linkou, Taoyuan City (TW)

(72) Inventors: Tsung-Ting Tsai, Taoyuan City (TW); Tse-Hao Huang, Taoyuan City (TW); Chien-Fu Chen, Taoyuan City (TW)

(73) Assignee: Chang Gung Memorial Hospital, Linkou, Taoyuan City (TW)

(21) Appl. No.: 16/100,471

(22) Filed: Aug. 10, 2018

(30) Foreign Application Priority Data
Mar. 31, 2018 (TW) 107111516

(51) Int. Cl. B01L 3/00; G01N 33/543

(52) U.S. Cl. CPC B01L 3/5023; G01N 33/54366; B01L 2300/0845; B01L 2300/0825; B01L 2300/0816; B01L 2300/069

(57) ABSTRACT

A detection device adapted for detecting an analyte in a sample includes a substrate having a top surface, a sample pad, a binding pad, a cellulose pad, a nitrocellulose membrane and an absorbent pad. The sample pad is for receiving the sample. The binding pad includes a main body and a first detecting reagent disposed on the main body and is adapted for specifically binding to the analyte. The cellulose pad has a first connecting end portion and a second connecting end portion. The nitrocellulose membrane includes a membrane body and a detection zone that includes a second detecting reagent adapted for specifically binding to the analyte. The absorbent pad connects to the membrane body.

### CROSS-REFERENCE TO RELATED APPLICATION

This application claims priority of Taiwanese Patent Application No. 107111516, filed on Mar. 31, 2018.

### FIELD

The disclosure relates to a detection device, and more particularly to an analyte detection device.

### BACKGROUND

Referring to FIG. 1, a conventional detection device 1 includes a substrate 11 extending in a longitudinal direction, a sample pad 12, a binding pad 13, a nitrocellulose membrane 14 and an absorbent pad 15. The sample pad 12, the binding pad 13, the nitrocellulose membrane 14 and the absorbent pad 15 are disposed on a top surface of the substrate 11 and are sequentially connected along the longitudinal direction.

The binding pad 13 is generally made from a glass fiber and includes a plurality of gold nanoparticle-labeled antibody. The nitrocellulose membrane 14 includes a control zone 141 and a detection zone 142 that are capable of developing color. During application of the detection device 1, a sample is loaded onto the sample pad 12, and then the sample flows into the bonding pad 13 through capillary phenomenon. If the sample contains analyte (e.g., antigen) that is specific to the gold nanoparticle-labeled antibody, the antigen will bind to the gold nanoparticle-labeled antibody, and the thus formed antigen-antibody complex then flows into the nitrocellulose membrane 14. Capturing of the antigen-antibody complex by an antibody immobilized in the detection zone 142 of the nitrocellulose membrane 14 allows color to be developed in the detection zone 142. Thus, the test result can be evaluated based on the color development in the detection zone 142.

However, since the time for the sample containing analyte staying in the bonding pad 13 of the conventional detection device 1 is short, the reaction time available for the gold nanoparticle-labeled antibody to bind to the antigen is therefore insufficient, thereby adversely affecting binding efficiency of the antigen-antibody complex. If the gold nanoparticle-labeled antibody is incompletely bound to the antigen, the accuracy of the detection device 1 would be reduced, particularly when the concentration of the antigen to be detected is low.

### SUMMARY

Therefore, an object of the disclosure is to provide a detection device that can alleviate at least one of the drawbacks of the prior art.

According to the disclosure, a detection device adapted for detecting an analyte in a sample includes a substrate, a sample pad, a binding pad, a cellulose pad, a nitrocellulose membrane and an absorbent pad. The substrate has a top surface. The sample pad, the binding pad, the cellulose pad, the nitrocellulose membrane and the absorbent pad are disposed on the top surface of the substrate. The sample pad is used for receiving the sample. The binding pad includes a main body and a first detecting reagent. The main body has a first end portion and a second end portion, and the first end portion is connected to the sample pad. The first detecting reagent is adapted for specifically binding to the analyte to forma complex, and is labeled with a detectable label and is disposed in the main body. The cellulose pad has a first connecting end portion connected to the second end portion of the main body, and a second connecting end portion. The nitrocellulose membrane includes a membrane body and a detection zone. The membrane body has a first membrane end portion connected to the second connecting end portion of the cellulose pad, and a second membrane end portion. The detection zone is provided in the membrane body and includes a second detecting reagent. The second detecting reagent is immobilized in the detection zone and is adapted for specifically binding to the analyte of the complex. In the detection zone, a detectable signal generated by the detectable label on the first detecting reagent is detected when the complex is captured by the second detecting reagent. The absorbent pad is connected to the second membrane end portion of the membrane body.

### BRIEF DESCRIPTION OF THE DRAWINGS

Other features and advantages of the present disclosure will become apparent in the following detailed description of the embodiment with reference to the accompanying drawings, of which:

FIG. 1 is a perspective view of a conventional detection device;

FIG. 2 is a perspective view of an embodiment of a detection device according to the present disclosure;

FIG. 3 is a photograph showing an analyte detection test result using conventional detection devices, comparative detection devices and detection devices of the embodiment; and

FIG. 4 is a bar chart showing color intensity of the detection zone of the detection devices shown in FIG. 3.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

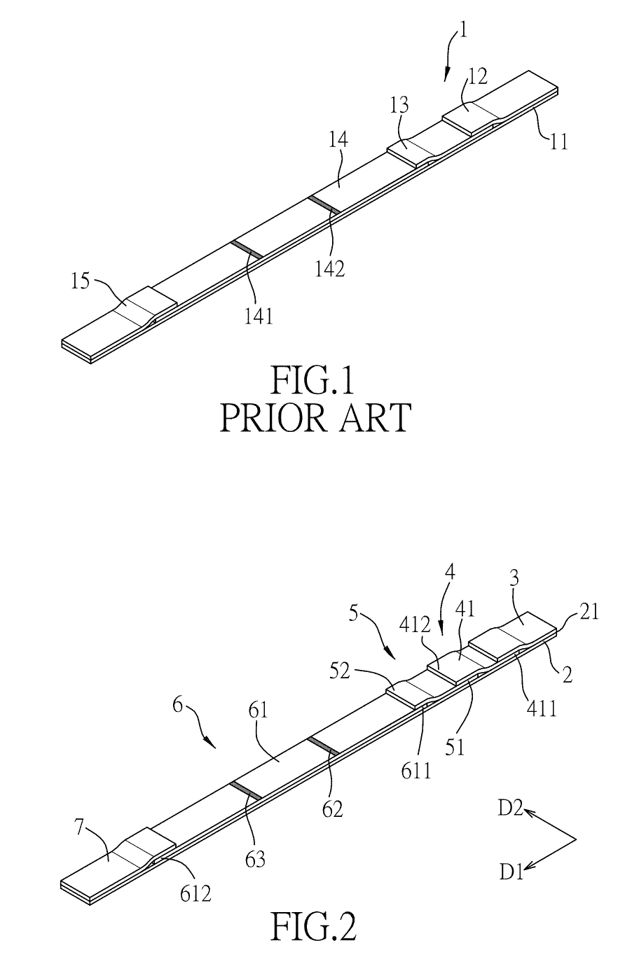

Referring to FIG. 2, an embodiment of a detection device of the pre sent disclosure is adapted for detecting an analyte in a sample and includes a substrate 2 having a top surface 21, and a sample pad 3 for receiving the sample, a binding pad 4, a cellulose pad 5, a nitrocellulose membrane 6 and an absorbent pad 7. In this embodiment, the substrate 2 has a strip shape and extends in a longitudinal direction (D1), and the sample pad 3, the binding pad 4, the cellulose pad 5, the nitrocellulose membrane 6 and the absorbent pad 7 are disposed on the top surface 21 of the substrate 2 and are sequentially connected along the longitudinal direction (D1).

Examples of material suitable for the sample pad 3 and the binding pad 4 of this disclosure may independently include, but are not limited to, glass fiber, polyester, and combinations thereof. In this embodiment, the sample pad 3 and the binding pad 4 are made of glass fiber.

The binding pad 4 includes a main body 41 and a first detecting reagent disposed on the main body 41. The main body 41 has a first end portion 411 and a second end portion 412, and the first end portion 411 and the second end portion 412 are respectively disposed at two opposite sides of the main body 41. In addition, the first end portion 411 is connected to the sample pad 3 and is disposed between the sample pad 3 and the top surface 21 of the substrate 2. To be specific, in this embodiment, a part of the sample pad 3 overlaps the first end portion 411 to form an overlapping area. The overlapping area has a width in a lateral direction (D2) transverse to the longitudinal direction (D1) that is substantially the same as widths of the sample pad 3 and the first end portion 411 of the binding pad 4.

The first detecting reagent is adapted for specifically binding to the analyte (e.g., an antigen) in the sample to form a complex and is labeled with a detectable label. Examples of the detectable label suitable to be used in this disclosure include, but are not limited to, a radioactive isotope label (such as ³H, ³¹P, ³⁵S, ¹⁴C, and ¹²⁵I), a hapten label (such as biotin/streptavidin and digoxigenin), a fluorescent label (such as CYE dyes, fluorescein isothiocyanate (FITC), rhodamine, phycoerythrin, coriphosphine-O (CPO), phycocyanin (PE), allophycocyanin (APC), o-phthaldehyde, fluorescamine and tandem dyes), a chemiluminescent label (such as gold-nanoparticle, luminol, luciferin, isoluminol, theromatic acridinium ester, imidazole, acridinium salt and oxalate ester), an enzymatic label (such as malate dehydrogenase, staphylococcal nuclease, delta-V-steroid isomerase, yeast alcohol dehydrogenase, alpha-glycerophosphate dehydrogenase, triose phosphate isomerase, horseradish peroxidase (HRP), alkaline phosphatase, asparaginase, glucose oxidase, β-galactosidase, ribonuclease, urease, catalase, glucose-VI-phosphate dehydrogenase, glucoamylase and acetylcholinesterase), a latex particle label (such as polystyrene, methacrylic acid polymer, acrylic acid polymer, styrene-acrylic acid copolymer, styrene-methacrylic acid copolymer, vinyl chloride-acrylic acid ester copolymer and polyvinyl acetate acrylate), a magnetic bead label (such as magnetic particles including iron, cobalt, nickel, ferrous oxide, ferrous hydroxide or other ferrous alloys), a quantum dot label, a nucleotide label, an epitope tag (such as T7, c-Myc, HA, VSV-G, HSV, FLAG, V5 and HIS), and combinations thereof.

According to this disclosure, when the analyte in the sample to be tested is an antigen, the first detecting reagent may be an antibody-based binding moiety.

As used herein, the term “antibody-based binding moiety” includes immunoglobulin molecules and immunologically active determinants of immunoglobulin molecules, e.g., molecules that contain an antigen-binding site that specifically binds to the antigen. The term “antibody-based binding moiety” is intended to include whole antibodies of any isotype (e.g., IgG, IgA, IgM, IgE, etc.), and fragments thereof.

According to this disclosure, the antibody-based binding moiety may include polyclonal, monoclonal or other purified preparations of antibodies and recombinant antibodies, and is further intended to include humanized antibodies, bi-specific antibodies, and chimeric molecules having at least one antigen-binding determinant derived from an antibody molecule.

For obtaining the binding pad 4, the first detecting reagent is dropped on the main body 41, followed by drying at 30° C. for 12 hours.

In this embodiment, the cellulose pad 5 is made of cellulose, and is connected to the binding pad 4. The celullose pad 5 has a first connecting end portion 51 and a second connecting end portion 52. The first connecting end portion 51 and the second connecting end portion 52 are respectively disposed on two opposite sides of the cellulose pad 5. The first connecting end portion 51 is connected to the second end portion 412 of the main body 41, and is disposed between the second end portion 412 and the top surface 21. To be specific, in this embodiment, the second end portion 412 of the main body 41 overlaps the first connecting end portion 51 of the cellulose pad 5 to form an overlapping area. The overlapping area has a width in the lateral direction (D2) transverse to the longitudinal direction (D1) that is substantially the same as widths of the second end portion 412 and the first connecting end portion 51. Furthermore, the binding pad 4 and the nitrocellulose membrane 6 are respectively disposed at two opposite sides of the cellulose pad 5.

The nitrocellulose membrane 6 includes a membrane body 61 and a detection zone 62 that is provided in the membrane body 61. The membrane body 61 has a first membrane end portion 611 connected to the second connecting end portion 52 of the cellulose pad 5, and a second membrane end portion 612. The first membrane end port ion 611 is disposed between the second connecting end portion 52 and the top surface 21, and the second connecting end portion 52 does not cover the detection zone 62. To be specific, in this embodiment, the second connecting end portion 52 of the cellulose pad 5 overlaps the first membrane end portion 611 of the membrane body 61 to form an overlapping area. The overlapping area has a width in the lateral direction (D2) transverse to the longitudinal direction (D1) that is substantially the same as widths of the second connecting end portion 52 and the first membrane end portion 611.

The detection zone 62 includes a second detecting reagent that is immobilized in the detection zone 62 and is adapted for specifically binding to the analyte of the complex. In the detection zone 62, a detectable signal generated by the detectable label on the first detecting reagent is detected when the complex is captured by the second detecting reagent. For preparing the detection zone 62, the second detecting reagent is dropped onto the detection zone 62, followed by drying at 30° C. for 12 hours. The nitrocellulose membrane 6 further includes a control zone 63 that is provided in the membrane body 61 and is separated from the detection zone 62. The control zone 63 includes a third detecting reagent that is immobilized in the control zone 63 and that is capable of specifically binding with the first detecting reagent. In the control zone 63, a detectable signal generated by the detectable label of the first detecting reagent is detected when the first detecting reagent is captured by the third detecting reagent. For preparing the control zone 63, the third detecting reagent is dropped onto the control zone 63, followed by drying at 30° C. for 12 hours. The control zone 63 is provided to determine whether the detection device is properly working.

In this embodiment, the first detecting reagent, the second detecting reagent and the third detecting reagent are respectively an antibody-based binding moiety when the analyte in the sample to be tested is an antigen.

In an exemplary embodiment, when the analyte in the sample is Protein A, the first detecting reagent that is labeled with the detectable label is gold nanoparticle-labeled rabbit polyclonal anti-Protein A IgG, the second detecting reagent immobilized in the detection zone 62 is rabbit polyclonal anti-Protein A IgG, and the third detecting reagent immobilized in the control zone 63 is goat anti-rabbit IgG.

The absorbent pad 7 is connected to the nitrocellulose membrane 6. The absorbent pad 7 can be used to absorb excess sample flowing from the nitrocellulose membrane 6. Examples of material suitable for ma king the absorbent pad 7 of this disclosure may include, but are not limited to, cotton pulp, cellulose, glass fiber, polyethylene and etc. In this embodiment, the absorbent pad 7 is made of cotton pulp.

In this embodiment, the absorbent pad 7 is connected to the second membrane end portion 612 of the membrane body 61. The second membrane end portion 612 is disposed between the absorbent pad 7 and the top surface 21 of the substrate 2. To be specific, in this embodiment, a portion of the absorbent pad 7 overlaps the second membrane end portion 612 of the membrane body 61 to form an overlapping area. The overlapping area has a width in the lateral direction (D2) transverse to the longitudinal direction (D1) that is substantially the same as widths of the portion of the absorbent pad 7 and the second membrane end portion 612. The absorbent pad 7 and the cellulose pad 5 are respectively disposed at opposite sides of the nitrocellulose membrane 6. The detection zone 62 is not covered by the absorbent pad 7.

When the embodiment of the present disclosure is used to determine the presence of the analyte in the sample, the sample is provided onto the sample pad 3, and the sample then flows into the binding pad 4 through capillary phenomenon. If the sample contains the analyte, the first detecting reagent will bind specifically to the analyte to form a complex, which then flows into the cellulose pad 5. The analyte and the first detecting reagent which moves along with the sample through capillary phenomenon may be further reacted with each other in the cellulose pad 5 to form the complex. The complex flows into the nitrocellulose membrane 6 and is captured by the second detecting reagent in the detection zone 62, so as to develop color in the detection zone 62. Therefore, a test result can be evaluated by the color development in the detection zone 62.

The first detecting reagent in the binding pad 4 is disposed and retained in the main body 41 after the drying process. The first detecting reagent should not be damaged or precipitated during the preparation of the binding pad 4. When the sample containing analyte flows into the binding pad 4, the first detecting reagent can be reacted with the analyte in the sample and is released from the main body 41, and the thus obtained complex can be rapidly flow out of the binding pad 4. Since glass fibers have an ideal adsorption capacity and releasing rate for the first detecting reagent, the main body 41 is generally made from the glass fibers. However, the loose structure of the glass fibers cause the sample to have a short staying time therein, and thus, the binding time between the first detecting reagent and the analyte in the sample may be too short to achieve complete binding effect.

To achieve complete binding effect, the cellulose pad 5 is provided in the detection device of this disclosure and is disposed downstream of the binding pad 4. Since the cellulose pad 5 has a relatively tight structure with small pore size, the flow rates of the sample and the first detecting reagent in the cellulose pad 5 may be reduced, and thus the staying time for the sample and the first detecting reagent in the cellulose pad 5 may be prolonged so that the binding efficiency between the analyte and the first detecting reagent may be improved and a sufficient amount of the complex may be formed. As such, the sensitivity of the detection device may be enhanced.

It is should be noted that, although tight structure of the cellulose material may reduce the flow rate and increase the staying time of the sample and the first detecting reagent, it would cause the first detecting reagent to be firmly retained therein and not to be easily released from the main body 41, which may adversely affect the detection test result. Therefore, cellulose is not suitable to be used as the material for making the binding pad 4.

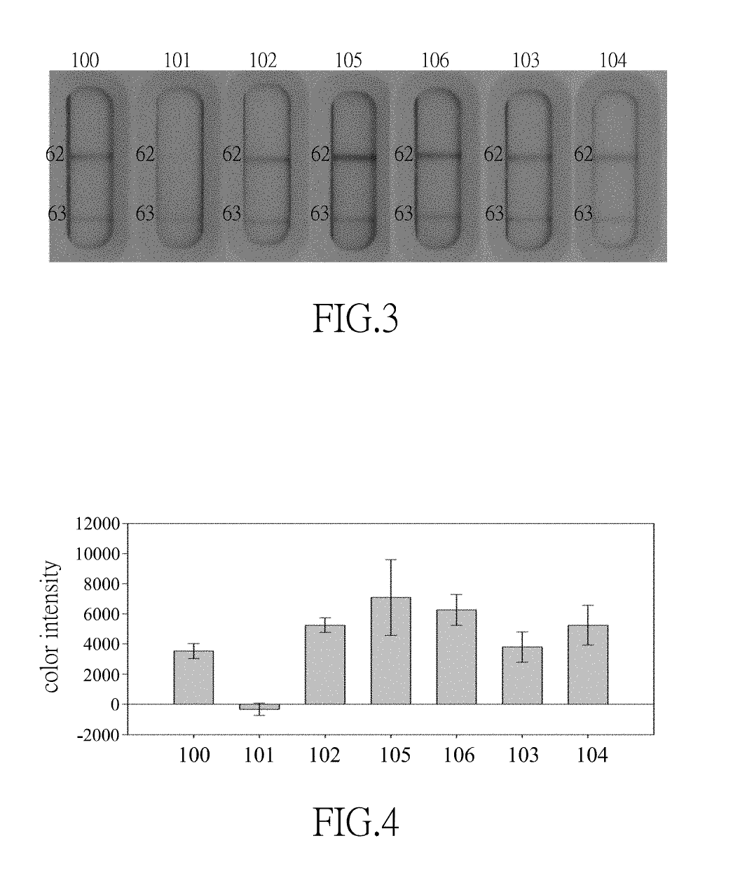

FIGS. 3 and 4 are photographs showing the results of an analyte detection test conducted using conventional detection devices, comparative detection devices and detection devices of the embodiment of this disclosure. The analyte in the sample used in this test is Protein A that is expressed on the cell surface of Staphylococcus aureus. The amount of the sample provided to each of the detecting devices is 0.2 mL and the concentration thereof is 10 ng/mL. Detection device 100 is the conventional detection device without the cellulose pad 5. Detection devices 105, 106 are detection devices of this embodiment respectively having 0.5 mm and 1 mm of the cellulose pads. Detection devices 101, 102 are the comparative detection devices with the cellulose pads being replaced by polyester pads, and the polyester pads of detection devices 101, 102 have thicknesses of 0.5 mm and 0.1 mm, respectively. Detection devices 103, 104 are the comparative detection devices with the cellulose pads 5 being replaced by glass fiber pads, and the glass fiber pads of detection devices 103, 104 have thicknesses of 0.6 mm and 0.2 mm, respectively.

From the results of analyte detection test of FIGS. 3 and 4, it is obvious that color intensity of the detection zone 62 of detection devices 105, 106 of this embodiment is greater than that of conventional detection devices 100, and that of comparative detection devices 101, 102, 103, 104 (about 1.5 to 2 times). For detection device 106, the cellulose pad is thicker than that of detection device 105, and thus a higher amount of the sample containing analyte can be loaded thereon. However, certain first detecting reagent may be trapped in the cellulose pad 5 and cannot flow into the nitrocellulose membrane 6, causing the color intensity of the detection zone 62 of detection device 106 to be relatively weak than that of the detection zone 62 of detection device 105. However, as shown in this embodiment, regardless of the thickness of the cellulose pad 5, the detection devices of this embodiment (i.e., detection devices 105 and 106) exhibit greater color intensity.

In summary, with the cellulose pad 5 of the detection device of this disclosure, the time taken for the sample containing analyte to flow into the nitrocellulose membrane 6 may be prolonged, allowing the first detecting reagent to have sufficient time to be reacted with the analyte in the sample. Therefore, the sensitivity and accuracy of the detection test result could be improved, particularly when the concentration of the analyte in the sample is low.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A detection device adapted for detecting an analyte in a sample, comprising:
a substrate having a top surface;
a sample pad disposed on said top surface of said substrate for receiving the sample;
a binding pad disposed on said top surface of said substrate and including
a main body having a first end portion and a second end portion, said first end portion being connected to said sample pad, and
a first detecting reagent adapted for specifically binding to the analyte to forma complex, said first detecting reagent being labeled with a detectable label and being disposed on said main body;
a cellulose pad disposed on said top surface of said substrate and having
a first connecting end portion connected to said second end portion of said main body, and
a second connecting end portion;
a nitrocellulose membrane disposed on said top surface of said substrate, and including
a membrane body that has a first membrane end portion connected to said second connecting end portion of said cellulose pad, and a second membrane end portion and
a detection zone that is provided in said membrane body, and that includes a second detecting reagent, said second detecting reagent being immobilized in said detection zone and being adapted for specifically binding to the analyte of the complex; and
an absorbent pad disposed on the top surface and connected to said second membrane end portion of said membrane body,
wherein, in said detection zone, a detectable signal generated by said detectable label on said first detecting reagent is detected when the complex is captured by said second detecting reagent.

2. The detection device of claim 1, wherein said binding pad and said nitrocellulose membrane are respectively disposed at two opposite sides of said cellulose pad.

3. The detection device of claim 1, wherein said first connecting end portion of said cellulose pad is disposed between said second end portion and said top surface.

4. The detection device of claim 1, wherein said first end portion and said second end portion are respectively disposed at two opposite sides of said main body.

5. The detection device of claim 1, wherein said cellulose pad and said absorbent pad are respectively disposed at two opposite sides of said nitrocellulose membrane.

6. The detection device of claim 1, wherein said first membrane end portion of said membrane body is disposed between said second connecting end portion and said top surface, and said second connecting end portion does not cover said detection zone of said nitrocellulose membrane.

7. The detection device of claim 1, wherein said first end portion of said main body is disposed between said sample pad and said top surface.

8. The biological detection device of claim 1, wherein said second membrane end portion of said membrane body is disposed between said absorbent pad and said top surface, and said absorbent pad does not cover said detection zone.

9. The detection device of claim 1, wherein said nitrocellulose membrane further includes a control zone provided in said membrane body and separated from said detection zone, said control zone including a third detecting reagent that is immobilized in said control zone and that is capable of specifically binding with said first detecting reagent, and wherein, in said control zone, a detectable signal generated by said detectable label on said first detecting reagent is detected when said first detecting reagent is captured by said third detecting reagent.

10. The detection device of claim 1, wherein said sample pad and said binding pad are respectively made of a material selected from the group consisting of glass fiber, polyester and combinations thereof.

11. The detection device of claim 1, wherein said absorbent pad is made of a material selected from the group consisting of cotton pulp, cellulose, glass fiber, polyethylene and combinations thereof.

12. The detection device of claim 1, wherein said first detecting reagent, said second detecting reagent, and said third detecting reagent are respectively an antibody-based binding moiety when the analyte is an antigen.
